(19) United States
(12) Patent Application Publication
(10) Pub. No.: US 2009/0253914 A1
(43) Pub. Date: Oct. 8, 2009

(54) PROCESS FOR THE PREPARATION OF ONIUM SALTS WITH DIALKYLPHOSPHATE, DIALKYLPHOSPHINATE OR (O-ALKYL)ALKYL- OR ALKYLPHOSPHONATE ANIONS HAVING A LOW HALIDE CONTENT

(75) Inventors: Nikolai (Mykola) Ignatyev, Duisburg (DE); Urs Welz-Biermann, Heppenheim (DE); Andriy Kucheryna, Kiev (UA); Helge Willner, Muelheim/Ruhr (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: MERCK PATENT GMBH, Darmstadt (DE)

(21) Appl. No.: 11/721,615

(22) PCT Filed: Nov. 18, 2005
(86) PCT No.: PCT/EP2005/012400

§ 371 Date: Jun. 13, 2007

(30) Foreign Application Priority Data
Dec. 14, 2004 (DE) 10 2004 060 075.9

(51) Int. Cl. C07D 213/20; C07D 233/58; C07F 9/09; C07C 211/13

(52) U.S. Cl. 546/347; 548/335.1; 558/87; 564/512

(57) ABSTRACT

The invention relates to a process for the preparation of onium salts with dialkylphosphate, dialkylphosphinate or (O-alkyl)alkyl- or alkylphosphonate anions by reaction of an onium halide with a triallyl phosphate, alkyl dialkylphosphinate, dialkyl alkylphosphonate or trialkylsilyl ester or mixed alkyl trialkylsilyl ester of phosphoric, dialkylphosphinic or alkylphosphonic acid.

The invention relates to a process for the preparation of onium salts with dialkylphosphate, dialkylphosphinate or (O-alkyl)alkyl- or alkylphosphonate anions by reaction of an onium halide with a trialkyl phosphate, alkyl dialkylphosphinate, dialkyl alkylphosphonate or trialkylsilyl ester or mixed alkyl trialkylsilyl ester of phosphoric, dialkylphosphinic or alkylphosphonic acid.

A large number of onium salts, including dialkylphosphates, dialkylphosphinates or phosphonates, can be used as ionic liquids. Due to their properties, ionic liquids represent an effective alternative to traditional volatile organic solvents for organic synthesis in modern research. The use of ionic liquids as novel reaction medium could furthermore be a practical solution both for solvent emission and also for problems in the reprocessing of catalysts.

Ionic liquids or liquid salts are ionic species which consist of an organic cation and a generally inorganic anion. They do not contain any neutral molecules and usually have melting points below 373 K. However, the melting point may also be higher without restricting the usability of the salts in all areas of application. Examples of organic cations are, inter alia, tetra-alkylammonium, tetraalkylphosphonium, N-alkylpyridinium, 1,3-dialkyl-imidazolium or trialkylsulfonium. Amongst a multiplicity of suitable anions, mention may be made, for example, of BF₄⁻, PF₆⁻, SbF₆⁻, NO₃⁻, CF₃SO₃⁻, (CF₃SO₂)₂N⁻, arylSO₃⁻, CF₃CO₂⁻, CH₃CO₂⁻ or Al₂Cl₇⁻.

A general method for the preparation of onium dialkylphosphates is, for example, alkylation of the organic base, i.e., for example, the amine, phosphine, guanidine or heterocyclic base, using a trialkyl phosphate, also disclosed by D. Corbridge, Phosphorus. An Outline of its Chemistry, Bio-chemistry and Technology, 2nd Edition, Elsevier, N.Y., 1980, or for phosphonium salts, disclosed by WO 04/094438. A general method for the preparation of onium dialkylphosphinates is disclosed by Jean, Bull. Soc. Chim. Fr. (1957), 783-785, or R. Jentzsch et al. J. Prakt. Chem. (1977), 319, 871-874.

A disadvantage of these methods is, however, that a substituent of the onium cation formed always corresponds to the corresponding alkyl group of the alkyl ester. If, for example, 1-butylimidazolium is reacted with trimethyl phosphate, 1-butyl-3-methylimidazolium dimethylphosphate is formed. However, asymmetrically substituted onium salts, i.e. salts in which the alkyl group of the ester employed is not a substituent of the onium salt formed, are desired.

Asymmetrical onium salts with dialkylphosphate, dialkylphosphinate, (O-alkyl)alkyl- or alkylphosphonate anions, as defined above, can also be prepared by a metathesis by reacting an onium halide with a corresponding alkali metal salt of the corresponding acid. However, the alkali metal halide formed, for example sodium chloride, has to be removed by an additional purification method. The contamination by halide ions, for example chloride ions, greater than 1000 ppm (0.1%), reduces the usability of the ionic liquid, in particular in the use for electrochemical processes. The technology is therefore of crucial importance in processes for the preparation of onium salts, in particular ionic liquids, in order that they can be synthesised with low impurity levels by the reaction per se or by the reaction procedure, and thus further expensive additional process steps during the synthesis are superfluous.

The object of the present invention was accordingly to provide an alternative process for the preparation of onium salts with dialkylphosphate, dialkylphosphinate, alkylphosphonate or (O-alkyl)alkylphosphonate anions having a low halide content which results in salts, preferably in asymmetrically substituted onium salts, of high purity in good yield and is also suitable for large-scale industrial production.

A process of this type is of course then also suitable for the preparation of symmetrically substituted onium salts.

The process according to the invention is likewise suitable for the preparation of onium salts with diarylphosphate, diarylphosphinate, arylphosphonate or mixed alkylarylphosphate, -phosphinate or -phosphonate anions. Aryl here describes, in particular, unsubstituted or substituted phenyl, where the substitution possibilities are described below for phenyl, and alkyl has a meaning described for the dialkylphosphates, dialkylphosphinates or alkylphosphonates.

The object is achieved by the process according to the invention since the ester employed alkylates the anion of the onium halide employed and not the organic onium cation. The alkyl halides formed as by-product are generally gases or very volatile compounds which can be removed from the reaction mixture without major engineering effort. Some of these by-products are themselves valuable materials for organic syntheses.

The invention therefore relates to a process for the preparation of onium salts with dialkylphosphate, dialkylphosphinate or (O-alkyl)alkyl- or alkylphosphonate anions by reaction of an onium halide with a trialkyl phosphate, alkyl dialkylphosphinate, dialkyl alkylphosphonate or trialkylsilyl ester or mixed alkyl trialkylsilyl ester of phosphoric, dialkylphosphinic or alkylphosphonic acid.

Suitable onium halides are phosphonium halides, thiouronium halides, guanidinium halides or halides with a heterocyclic cation, where the halides can be selected from the group chlorides, bromides or iodides. Chlorides or bromides are preferably employed in the process according to the invention. For the preparation of thiouronium salts, thiouronium iodides are preferably employed.

The onium halides are generally commercially available or can be prepared by synthetic methods as known from the literature, for example in the standard works, such as Houben-Weyl, Methoden der organischen Chemie [Methods of Organic Chemistry], Georg-Thieme-Verlag, Stuttgart, or Richard C. Larock, Comprehensive Organic Transformations, 2nd Edition, Wiley-VCH, New York, 1999. Use can also be made here of variants known per se which are not mentioned here in greater detail.

Phosphonium halides can be described, for example, by the formula (1)

[PR₄]⁺Hal⁻ (1),

where
Hal denotes Cl, Br or I and
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms, where one or more R may be partially or fully substituted by —F, but where all four or three R must not be fully substituted by F,
and where, in the R, one or two non-adjacent carbon atoms which are not in the α- or ω-position may be replaced by atoms and/or atom groups selected from the group —O—, —S—, —S(O)— or —SO₂—.

However, compounds of the formula (1) in which all four or three substituents R are fully substituted by halogens, for example tris(trifluoromethyl)methylphosphonium chloride, tetra(trifluoromethyl)phosphonium chloride or tetra(nonafluorobutyl)phosphonium chloride, are excluded.

Thiouronium halides can be described, for example, by the formula (2)

[(R¹R²N)—C(═SR⁷)(NR³R⁴)]⁺Hal⁻ (2)

and guanidinium halides can be described, for example, by the formula (3)

[C(NR¹R²)((NR³R⁴)(NR⁵R⁶)]⁺Hal⁻ (3),

where
Hal denotes Cl, Br or I and
R′ to R⁷ each, independently of one another, denote hydrogen or CN, where hydrogen is excluded for R⁷,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms,
which may be substituted by alkyl groups having 1-6 C atoms,
where one or more of the substituents R¹ to R⁷ may be partially or fully substituted by —F, but where all substituents on an N atom must not be fully substituted by F,
where the substituents R¹ to R⁷ may be bonded to one another in pairs by a single or double bond
and where, in the substituents R¹ to R⁷, one or two non-adjacent carbon atoms which are not bonded directly to the heteroatom and are not in the ω-position may be replaced by atoms and/or atom groups selected from the group —O—, —S—, —S(O)— or —SO₂—.
Halides with a heterocyclic cation can be described, for example, by the formula (4)

[HetN]⁺Hal⁻ (4)

where
Hal denotes Cl, Br or I and
HetN⁺ denotes a heterocyclic cation selected from the group

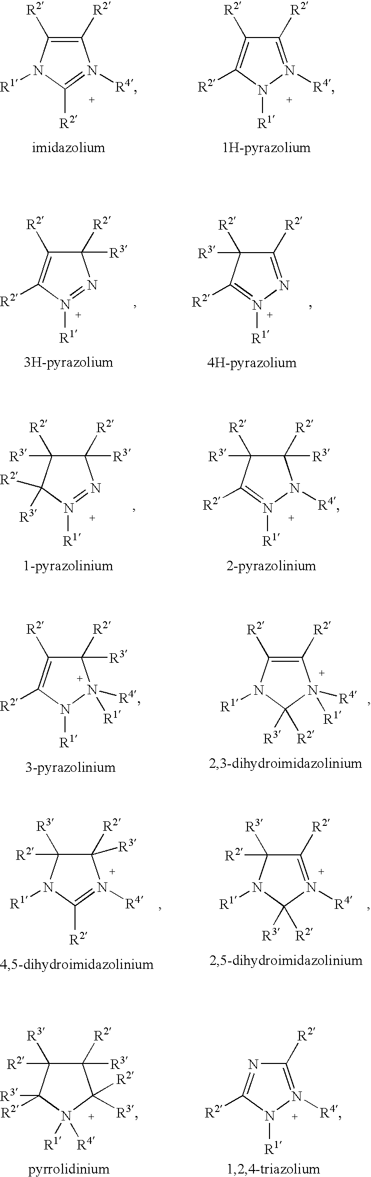

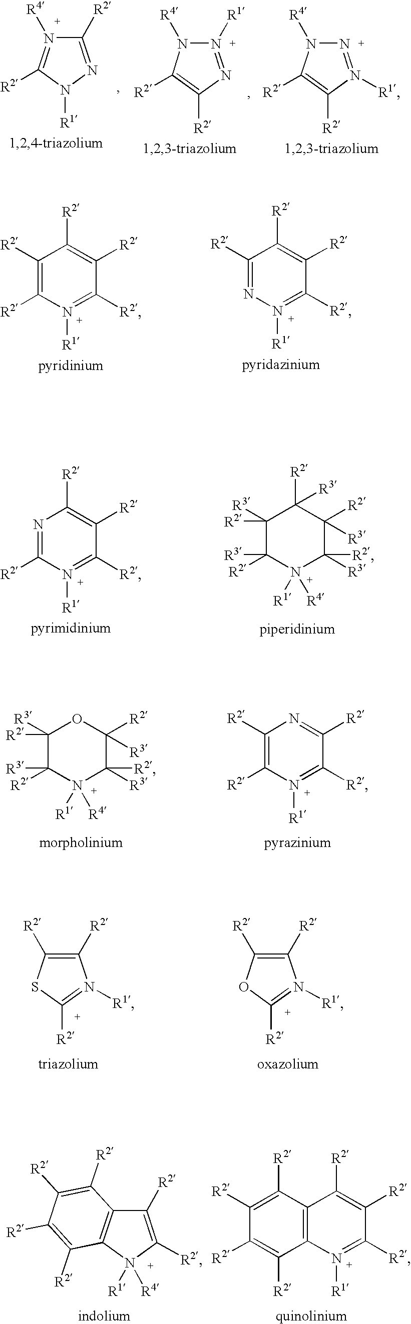

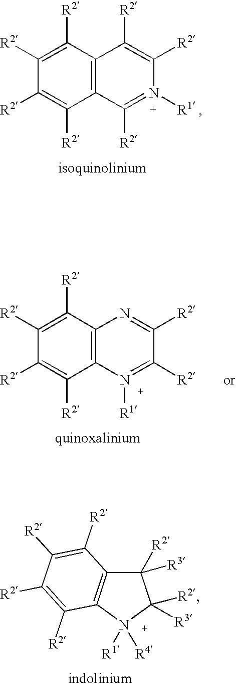

where the substituents
R^{1′} to R^{4′} each, independently of one another, denote hydrogen or CN,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
dialkylamino having alkyl groups having 1-4 C atoms, but which is not bonded to the heteroatom of the heterocycle,
saturated, partially or fully unsaturated cycloalkyl having 3-7 C atoms,
which may be substituted by alkyl groups having 1-6 C atoms, or aryl-C₁-C₆-alkyl,
where the substituents R^{1′} and R^{4′} may be partially or fully substituted by F, but where R^{1″} and R^{4′} cannot simultaneously be CN or fully substituted by F,
where the substituents R^{2′} and R^{3′} may be partially or fully substituted by halogens or partially substituted by NO₂ or CN
and where, in the substituents R^{1′} to R^{4′}, one or two non-adjacent carbon atoms which are not bonded directly to the heteroatom and are not in the ω-position may be replaced by atoms and/or atom groups selected from the group —O—, —S—, —S(O)— or —SO₂—.

For the purposes of the present invention, fully unsaturated substituents are also taken to mean aromatic substituents.

In accordance with the invention, suitable substituents R and R¹ to R⁷ of the compounds of the formulae (1) to (3), besides hydrogen, are preferably: C₁- to C₂₀-, in particular C₁- to C₁₄-alkyl groups, and saturated or unsaturated, i.e. also aromatic, C₃- to C₇-cycloalkyl groups, which may be substituted by C₁- to C₆-alkyl groups, in particular phenyl.

However, the substituents R and R¹ to R⁷ may likewise be substituted by further functional groups, for example by CN, SO₂R′, SO₂OR′ or COOR′, R′ denotes non-fluorinated or partially fluorinated C₁- to C₆-alkyl, C₃- to C₇-cycloalkyl, unsubstituted or substituted phenyl.

The substituents R in the compounds of the formula (1) may be identical or different here. Preferably, three substituents in formula (1) are identical and one substituent is different.

The substituent R is particularly preferably methyl, ethyl, isopropyl, propyl, butyl, sec-butyl, pentyl, hexyl, octyl, decyl or tetradecyl.

Up to four substituents of the guanidinium cation [C(NR¹R²)(NR³R⁴)(NR⁵R⁶)]⁺ may also be connected in pairs in such a way that mono-, bi- or polycyclic cations are formed.

Without restricting generality, examples of such guanidinium cations are:

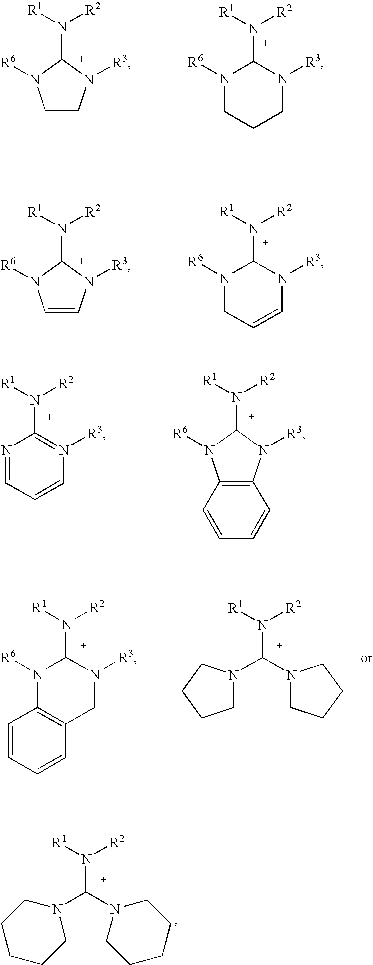

where the substituents R¹ to R³ and R⁶ may have an above-mentioned or particularly preferred meaning.

The carbocycles or heterocycles of the above-mentioned guanidinium cations may optionally also be substituted by C₁- to C₆-alkyl, C₁- to C₆-alkenyl, NO₂, F, Cl, Br, I, C₁-C₆-alkoxy, SCF₃, SO₂CH₃, SO₂CF₃, COOR″, SO₂NR″₂, SO₂X′, SO₃R″ or substituted or unsubstituted phenyl, where X′ and R″ have a meaning indicated above or below.

Up to four substituents of the thiouronium cation [(R¹R²N)—C(═SR⁷)—(NR³R⁴)]⁺ may also be connected in pairs in such a way that mono-, bi- or polycyclic cations are formed.

Without restricting generality, examples of such cations are indicated below:

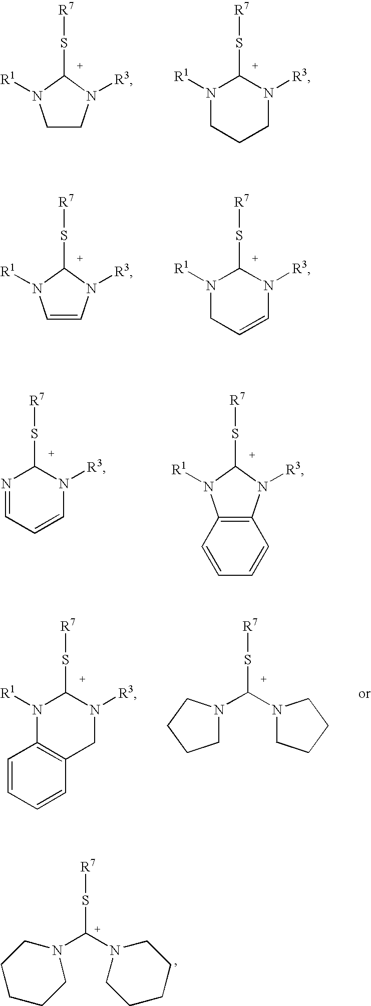

where the substituents R¹, R³ and R⁷ may have an above-mentioned or particularly preferred meaning.

The carbocycles or heterocycles of the above-mentioned guanidinium cations may optionally also be substituted by C₁- to C₆-alkyl, C₁- to C₆-alkenyl, NO₂, F, Cl, Br, I, C₁-C₆-alkoxy, SCF₃, SO₂CH₃, SO₂CF₃, COOR″, SO₂NR″₂, SO₂X′, SO₃R″ or substituted or unsubstituted phenyl, where X′ and R″ have a meaning indicated above or below.

The C₁-C₁₄-alkyl group is, for example, methyl, ethyl, isopropyl, propyl, butyl, sec-butyl or tert-butyl, furthermore also pentyl, 1-, 2- or 3-methylbutyl, 1,1-, 1,2- or 2,2-dimethylpropyl, 1-ethylpropyl, hexyl, heptyl, octyl, nonyl, decyl, undecyl, dodecyl, tridecyl or tetradecyl, optionally perfluorinated, for example as difluoromethyl, trifluoromethyl, pentafluoroethyl, heptafluoropropyl or nonafluorobutyl.

A straight-chain or branched alkenyl having 2 to 20 C atoms, where a plurality of double bonds may also be present, is, for example, vinyl, allyl, 2- or 3-butenyl, isobutenyl, sec-butenyl, furthermore 4-pentenyl, isopentenyl, hexenyl, heptenyl, octenyl, —C₉H₁₇, —C₁₀H₁₉ to —C₂₀H₃₉, preferably allyl, 2- or 3-butenyl, isobutenyl, sec-butenyl, furthermore preferably 4-pentenyl, isopentenyl or hexenyl.

A straight-chain or branched alkynyl having 2 to 20 C atoms, where a plurality of triple bonds may also be present, is, for example, ethynyl, 1- or 2-propynyl, 2- or 3-butynyl, furthermore 4-pentynyl, 3-pentynyl, hexynyl, heptynyl, octynyl, —C₉H₁₅, —C₁₀H₁₇ to —C₂₀H₃₇, preferably ethynyl, 1- or 2-propynyl, 2- or 3-butynyl, 4-pentynyl, 3-pentynyl or hexynyl.

Aryl-C₁-C₆-alkyl denotes, for example, benzyl, phenylethyl, phenylpropyl, phenylbutyl, phenylpentyl or phenylhexyl, where both the phenyl ring and also the alkylene chain may be partially or fully substituted as described above by halogens, in particular —F and/or —Cl, or partially substituted by —NO₂, particularly preferably benzyl or phenylpropyl. However, the phenyl ring or also the alkylene chain may likewise be substituted by further functional groups, for example by CN, SO₂R′, SO₂OR′ or COOR′, where R′=non- or partially fluorinated C₁- to C₆-alkyl, C₃- to C₇-cycloalkyl, unsubstituted or substituted phenyl.

Unsubstituted saturated or partially or fully unsaturated cycloalkyl groups having 3-7 C atoms are therefore cyclopropyl, cyclobutyl, cyclopentyl, cyclohexyl, cycloheptyl, cyclopentenyl, cyclopenta-1,3-dienyl, cyclohexenyl, cyclohexa-1,3-dienyl, cyclohexa-1,4-dienyl, phenyl, cycloheptenyl, cyclohepta-1,3-dienyl, cyclohepta-1,4-dienyl or cyclohepta-1,5-dienyl, each of which may be substituted by C₁- to C₆-alkyl groups, where the cycloalkyl group or the C₁- to C₆-alkyl-substituted cycloalkyl group may in turn also be substituted by halogen atoms, such as F, Cl, Br or I, in particular F or Cl, or NO₂. However, the cycloalkyl groups may likewise be substituted by further functional groups, for example by CN, SO₂R′, SO₂OR′ or COOR′. R′ here has a meaning defined above.

In the substituents R, R¹ to R⁶ or R^{1′} to R^{4′}, one or two non-adjacent carbon atoms which are not bonded in the α-position to the heteroatom or in the ω-position may also be replaced by atoms and/or atom groups selected from the group —O—, —S—, —S(O)— or —SO₂—.

Without restricting generality, examples of substituents R, R¹ to R⁶ and R^{1′} to R^{4′} modified in this way are:

—OCH₃, —OCH(CH₃)₂, —CH₂OCH₃, —CH₂—CH₂—O—CH₃, —C₂H₄OCH(CH₃)₂, —C₂H₄C₂H₅, —C₂H₄SCH(CH₃)₂, —S(O)CH₃, —SO₂CH₃₁—SO₂C₆H₅, —SO₂C₃H₇, —SO₂CH(CH₃)₂, —SO₂CH₂CF₃, —CH₂SO₂CH₃, —O—C₄H₈—O—C₄H₉, —CF₃, —C₂F₅, —C₃F₇, —C₄F₉, —CF₂CF₂H, —CF₂CHFCF₃₁—CF₂CH(CF₃)₂, —C₂F₄N(C₂F₅)C₂F₅, —CHF₂, —CH₂CF₃, —C₂F₂H₃, —C₃H₆, —CH₂C₃F₇, —CH₂C(O)OCH₃, —CH₂C₆H₅ or —C(O)C₆H₅

R′ is C₃- to C₇-cycloalkyl, for example cyclopropyl, cyclobutyl, cyclopentyl, cyclohexyl or cycloheptyl.

In R′, substituted phenyl denotes phenyl which is substituted by C₁- to C₆-alkyl, C₁- to C₆-alkenyl, NO₂, F, Cl, Br, I, C₁-C₆-alkoxy, SCF₃, SO₂CF₃, COOR″, SO₂X′, SO₂NR″₂ or SO₃R″, where X′ denotes F. Cl or Br and R″ denotes a non- or partially fluorinated C₁- to C₆-alkyl or C₃- to C₇-cycloalkyl, as defined for R′, for example o-, m- or p-methylphenyl, o-, m- or p-ethylphenyl, o-, m- or p-propylphenyl, o-, m- or p-isopropylphenyl, o-, m- or p-tert-butylphenyl, o-, m- or p-nitrophenyl, o-, m- or p-methoxyphenyl, o-, m- or p-ethoxyphenyl, o-, m-, p-(trifluoromethyl)phenyl, o-, m-, p-(trifluoromethoxy)phenyl, o-, m-, p-(trifluoromethylsulfonyl)phenyl, o-, m- or p-fluorophenyl, o-, m- or p-chlorophenyl, o-, m- or p-bromophenyl, o-, m- or p-iodophenyl, further preferably 2,3-, 2,4-, 2,5-, 2,6-, 3,4- or 3,5-dimethylphenyl, 2,3-, 2,4-, 2,5-, 2,6-, 3,4- or 3,5-difluorophenyl, 2,3-, 2,4-, 2,5-, 2,6-, 3,4- or 3,5-dichlorophenyl, 2,3-, 2,4-, 2,5-, 2,6-, 3,4- or 3,5-dibromophenyl, 2,3-, 2,4-, 2,5-, 2,6-, 3,4- or 3,5-dimethoxyphenyl, 5-fluoro-2-methylphenyl, 3,4,5-trimethoxyphenyl or 2,4,5-trimethylphenyl.

The substituents R¹ to R⁷ are each, independently of one another, preferably a straight-chain or branched alkyl group having 1 to 10 C atoms. The substituents R¹ and R², R³ and R⁴ and R⁵ and R⁶ in compounds of the formulae (2) and (3) may be identical or different here.

R¹ to R⁷ are particularly preferably each, independently of one another, methyl, ethyl, n-propyl, isopropyl, n-butyl, sec-butyl, phenyl or cyclohexyl, very particularly preferably methyl, ethyl, n-propyl, isopropyl or n-butyl.

In accordance with the invention, suitable substituents R^{1′} to R^{4′} of compounds of the formula (4), besides hydrogen, are preferably: C₁- to C₂₀-, in particular C₁- to C₁₂-alkyl groups, and saturated or unsaturated, i.e. also aromatic, C₃- to C₇-cycloalkyl groups, which may be substituted by C₁- to C₆-alkyl groups, in particular phenyl or aryl-C₁-C₆-alkyl.

The substituents R^{1′} and R^{4′} are each, independently of one another, particularly preferably methyl, ethyl, isopropyl, propyl, butyl, sec-butyl, pentyl, hexyl, octyl, decyl, cyclohexyl, phenyl, phenylpropyl or benzyl. They are very particularly preferably methyl, ethyl, n-butyl or hexyl. In pyrrolidinium, piperidinium or indolinium compounds, the two substituents R^{1′} and R^{4′} are preferably different.

The substituent R^{2′} or R^{3′} is in each case, independently of one another, in particular hydrogen, methyl, ethyl, isopropyl, propyl, butyl, sec-butyl, tertbutyl, cyclohexyl, phenyl or benzyl. R^{2′} is particularly preferably hydrogen, methyl, ethyl, isopropyl, propyl, butyl, sec-butyl or tert-butyl. R^{2′} and R^{3′} are very particularly preferably hydrogen or methyl.

The alkyl groups as substituents R and R¹ to R⁶ and R^{1′} and R^{4′} of the heterocyclic cations of the formula (4) are preferably different from the alkyl group of the corresponding ester, trialkylsilyl ester or mixed alkyl trialkylsilyl ester of phosphoric, dialkylphosphinic or alkylphosphonic acid employed.

The onium dialkylphosphate, onium dialkylphosphinate, onium (O-alkyl)alkylphosphonate or onium alkylphosphonate prepared in accordance with the invention may, however, also have alkyl groups in the cation which are identical with the alkyl group in the ester, but were not introduced in accordance with the invention by alkylation. The focus is then on the simple reaction procedure and the particularly low halide content in the end product.

HetN⁺ of the formula (4) is preferably

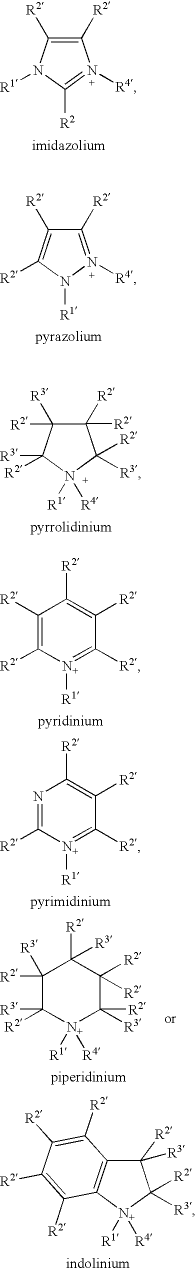

where the substituents R^{1′} to R^{4′} each, independently of one another, have a meaning described above.

HetN⁺ is particularly preferably imidazolium, pyrrolidinium or pyridinium, as defined above, where the substituents R^{1′} to R^{4′} each, independently of one another, have a meaning described above.

The ester of a phosphoric, phosphinic or phosphonic acid employed is preferably a corresponding ester having straight-chain or branched alkyl groups having 1-8 C atoms, preferably having 1-4 C atoms, which are in each case independent of one another. The alkyl groups of the ester are preferably identical.

The alkyl esters of a phosphoric, phosphinic or phosphonic acid employed are generally commercially available or can be prepared by synthetic methods as known from the literature, for example in the standard works, such as Houben-Weyl, Methoden der organischen Chemie [Methods of Organic Chemistry], Georg-Thieme-Verlag, Stuttgart, or Richard C. Larock, Comprehensive Organic Transformations, 2nd Edition, Wiley-VCH, New York, 1999. Use can also be made here of variants known per se which are not mentioned here in greater detail.

Examples of trialkyl phosphates are trimethyl phosphate, triethyl phosphate, tripropyl phosphate, tributyl phosphate, tripentyl phosphate, trihexyl phosphate, triheptyl phosphate or trioctyl phosphate. Particular preference is given to the use of trimethyl phosphate or triethyl phosphate.

Examples of dialkylphosphinic acid esters are methyl dimethylphosphinate, ethyl dimethylphosphinate, methyl bis(trifluoromethyl)phosphinate, methyl diethylphosphinate, ethyl diethylphosphinate, methyl bis(pentafluoroethyl)phosphinate, ethyl bis(pentafluoroethyl)phosphinate or methyl bis(nonafluorobutyl)phosphinate.

Examples of dialkyl alkylphosphonates are dimethyl methylphosphonate, diethyl methylphosphonate, dimethyl ethylphosphonate, dimethyl pentafluoroethylphosphonate, dimethyl trifluoromethylphosphonate, diethyl ethylphosphonate or dimethyl nonafluorobutylphosphonate.

Trialkylsilyl esters or mixed alkyl trialkylsilyl esters of phosphoric acid, dialkylphosphinic acid or alkylphosphonic acid which can be employed are tris(trialkylsilyl) phosphate, bis(trialkylsilyl) alkyl phosphate, trialkylsilyl dialkyl phosphate, trialkylsilyl dialkylphosphinate, trialkylsilyl O-alkyl alkylphosphonate or bis(trialkylsilyl) alkylphosphonate, where the alkyl groups may be linear or branched having 1 to 8 C atoms, preferably having 1 to 4 C atoms. The alkyl groups of the trialkylsilyl group are preferably identical and have 1 to 4 C atoms.

Examples of the above-mentioned esters are tris(trimethylsilyl) phosphate, bis(trimethylsilyl)methyl phosphate, bis(trimethylsilyl)ethyl phosphate, trimethylsilyl dimethyl phosphate, trimethylsilyl dimethylphosphinate, triethylsilyl diethylphosphinate, trimethylsilyl bis(pentafluoroethyl)phosphinate, bis(trimethylsilyl)methylphosphonate, bis(trimethylsilyl) pentatluoroethylphosphonate, bis(trimethylsilyl) pentafluoroethylphosphonate or bis(triethylsilyl) nonafluorobutylphosphonate.

The trialkylsilyl esters or mixed esters of a phosphoric, phosphinic or phosphonic acid employed, as described above, are generally commercially available or can be prepared by synthetic methods as known from the literature, for example in the standard works, such as Houben-Weyl, Methoden der organischen Chemie [Methods of Organic Chemistry], Georg-Thieme-Verlag, Stutgart, or Richard C. Larock, Comprehensive Organic Transformations, 2nd Edition, Wiley-VCH, New York, 1999. Use can also be made here of variants known per se which are not mentioned here in greater detail.

A general scheme summarises the process according to the invention:

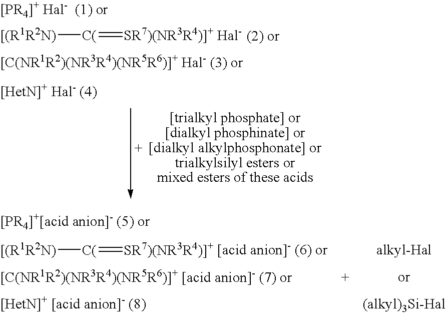

The substituents R, R¹ to R⁷ and HetN⁺ of the compounds of the formulae (1) to (8) correspond to the meanings as described above. [Acid anion]⁻ denotes the corresponding anion from the ester employed after removal of an alkyl group, for example [(alkyl-O)₂P(O)]⁻, [(alkyl)₂P(O)O]⁻ or [(alkyl-O)(alkyl)P(O)O]⁻.

The reaction is carried out in accordance with the invention at temperatures between 200 and 100° C., preferably at 80° to 100°, particularly preferably at 100° C., if alkyl esters of the corresponding acids are employed. If the trialkylsilyl esters or mixed esters of the acids are employed, the reaction is carried out at between 0° C. and 30° C., preferably at room temperature. No solvent is required. However, it is also possible to employ solvents, for example dimethoxyethane, acetonitrile, acetone, tetrahydrofuran, dimethylformamide, dimethyl sulfoxide, dioxane, propionitrile or mixtures thereof.

The reaction is carried out with a maximum excess of up to 20% or an equimolar amount of the corresponding ester of phosphoric, phosphinic or phosphonic acid.

The method according to the invention can also be used for the purification of halide-containing onium salts with dialkylphosphate, dialkylphosphinate, alkylphosphonate or (O-alkyl)alkylphosphonate anions.

The invention also relates to the starting materials of the trialkylsilyl esters of dialkylphosphinic acid, in particular (C₂F₅)₂P(O)OSi(alkyl)₃, (C₃F₇)₂P(O)—OSi(alkyl)₃ and (C₄F₉)₂P(O)OSi(alkyl)₃, where the alkyl groups of the trialkylsilyl group can have 1 to 4 C atoms. The alkyl groups of the trialkylsilyl group are preferably identical.

Particularly preferred trialkylsilyl esters are (C₂F₅)₂P(O)OSi(CH₃)₃, (C₂F₅)₂P(O)OSi(C₂H₅)₃, (C₃F₇)₂P(O)OSi(CH₃)₃, (C₃F₇)₂P(O)OSi(C₂H₅)₃, (C₄F₉)₂P(O)OSi(CH₃)₃ or (C₄F₉)₂P(O)OSi(C₂H₅)₃.

These compounds are, in particular, excellent silylating reagents, independently of the process according to the invention.

A known trialkylsilyl ester is (CF₃)₂P(O)OSi(CH₃)₃, but this compound is difficult to prepare and is unstable since the F₃C—P bond is labile.

Even without further comments, it is assumed that a person skilled in the art will be able to utilise the above description in the broadest scope. The preferred embodiments and examples should therefore merely be regarded as descriptive disclosure which is absolutely not limiting in any way.

It goes without saying for the person skilled in the art that substituents in the compounds mentioned above and below, such as, for example, H, N, O, Cl or F, can be replaced by the corresponding isotopes.

The NMR spectra were measured on solutions in deuterated solvents at 20° C. on a Bruker ARX 400 spectrometer with a 5 mm ¹H/BB broadband head with deuterium lock, unless indicated in the examples. The measurement frequencies of the various nuclei are: ¹H: 400, 13 MHz and ¹⁹F: 376.50 MHz. ³¹P spectra were measured on a Bruker Avance 250 spectrometer with the measurement frequency 101.26 MHz. The referencing method is indicated separately for each spectrum or each data set.

### EXAMPLE 1

### Synthesis of 1-butyl-3-methylimidazolium dimethylphosphate

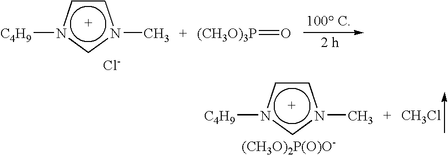

A mixture of 3.25 g (18.6 mmol) of 1-butyl-3-methylimidazolium chloride and 2.61 g (18.6 mmol) of trimethyl phosphate is heated at an oil-bath temperature of 100° C. for two hours. The residue is dried at 100° C. (oil-bath temperature) and a vacuum of 13.3 Pa for one hour, giving 4.91 g of 1-butyl-3-methylimidazolium dimethylphosphate in virtually quantitative yield.

¹H NMR (reference: TMS; CD₃CN), ppm: 0.88 t (CH₃); 1.27 m (CH₂); 1.79 m (CH₂); 3.37 d (OCH₃); 3.87 s (CH₃); 4.18 t (CH₂); 7.57 m (CH); 7.60 m (CH); 10.03 br. 5. (CH); ³J_{H,H}=7.4 Hz; ³J_{H,H}=7.2 Hz; ³J_{P,H}=10.4 Hz.

³¹P {¹H} NMR (reference: 85% H₃PO₄—external; CD₃CN), ppm: 1.71.

### EXAMPLE 2

### Synthesis of Tetraethylphosphonium Dimethylphosphate

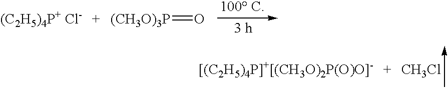

A mixture of 0.50 g (2.74 mmol) of tetraethylphosphonium chloride and 0.46 g (3.28 mmol) of trimethyl phosphate is heated at 100° C. for 3 hours, The residue is subsequently treated at 10000 for 30 minutes under a vacuum of 13.3 Pa, giving 0.74 g of tetraethylphosphonium dimethylphosphate. The yield is virtually quantitative.

M.p. 48-49° C.

¹H NMR (reference: TMS; CD₃CN), ppm: 1.19 d,t (4CH₃); 2.26 m (4CH₂); 3.37 d (20CH₃); ³J_{H,P}=10.3 Hz; ³J_{H,P}=18.0 Hz; ³J_{H,H}=7.7 Hz. ³¹P NMR (reference: 85% H₃PO₄—external; CD₃CN), ppm: 0.4 hep (1P); 39.5 m (1P).

### EXAMPLE 3

### Synthesis of 1-butyl-3-methylimidazolium bis(pentafluoroethyl)phosphinate

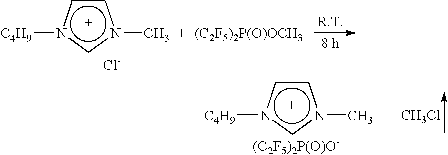

A mixture of 0.693 g (3.97 mmol) of 1-butyl-3-methylimidazolium chloride and 1.256 g (3.97 mmol) of methyl bis(pentafluoroethyl)phosphinate is stirred at room temperature for 8 hours. NMR measurements confirm the completeness of the reaction. The residue is dried for 30 minutes at 90° C. under a vacuum of 13.3 Pa, giving 1.74 g of 1-butyl-3-methylimidazolium bis(pentafluoroethyl)phosphinate as a liquid. The yield is virtually quantitative,

¹H NMR (reference: TMS; CD₃CN), ppm: 0.93 t (CH₃); 1.32 m (CH₂), 1.81 m (CH₂), 3.84 s (CH₃); 4.15 t (CH₂); 7.40 m (CH); 7.45 m (CH); 8.84 br. S. (CH); ³J_{H,H}=7.4 Hz; ³J_{H,H}=7.3 Hz.

¹⁹F NMR (reference: CCl₃F—internal; CD₃CN), ppm: −80.2 s (2CF₃); −124.9 d (2CF₂); ²J_{P,F}=66 Hz.

³¹p NMR (reference: 85% H₃PO₄— external; OD₃CN), ppm: −2.5 quin.; ²J_{P,F}=66 Hz.

### EXAMPLE 4

### Synthesis of 1-butyl-3-methylimidazolium bis(pentafluoroethyl)phosphinate

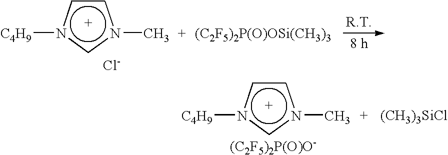

A mixture of 0.506 g (2.30 mmol) of 1-butyl-3-methylimidazolium chloride and 1.084 g (2.30 mmol) of trimethylsilyl bis(pentafluoroethyl)phosphinate is stirred at room temperature for 8 hours, NMR measurements confirm the completeness of the reaction. The residue is dried for 30 minutes at 90° C. and 13.3 Pa, giving 1.275 g of 1-butyl-3-methylimidazolium bis(pentafluoroethyl)phosphinate as a liquid. The yield is virtually quantitative.

The NMR spectra correspond to Example 3.

### EXAMPLE 5

### Synthesis of N,N, N′,N′-tetramethyl-N″-ethylguanidinium bis(pentafluoroethyl)phosphinate

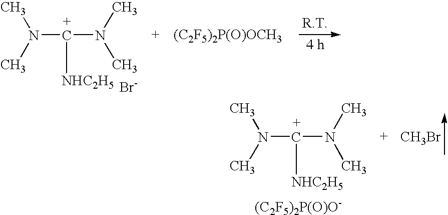

A mixture of 0.90 g (4.015 mmol) of N,N,N′,N′-tetramethyl-N″-ethylguanidinium bromide and 1.27 g (4.018 mmol) of methyl bis(pentafluoroethyl)phosphinate is stirred at room temperature for 4 hours, NMR measurements confirm the completeness of the reaction. The residue is dried for 30 minutes at 9000 under a vacuum of 13.3 Pa, giving 1.77 g of N,N,N′,N′-tetramethyl-N″-ethylguanidinium bis(pentafluoroethyl)phosphinate. The yield is virtually quantitative.

M.p.: 50-52° C.

¹H NMR (reference: TMS; CD₃CN), ppm: 1.13 t (CH₃); 2.87 br.s; 2.89 br.s; 2.92 s (4CH₃); 3.21 m (CH₂); 7.14 br.s (NH); ³J_{H,H}=7.1 Hz.

¹⁹F NMR (reference: CCl₃F —internal; CD₃CN), ppm: −80.2 S (2CF₃); −124.9 d (2CF₂); ²J_{P,F}=67 Hz.

³¹P NMR (reference: 85% H₃PO₄—external; CD₃CN), ppm: −2.8 quin.;

²J_{P,F}=67 Hz.

### EXAMPLE 6

### Synthesis of 1-butylpyridinium bis(pentafluoroethyl)phosphinate

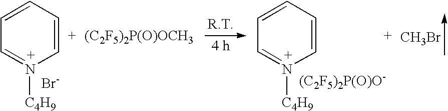

A mixture of 0.83 g (3.84 mmol) of 1-butylpyridinium bromide and 1.22 g (3.86 mmol) of methyl bis(pentafluoroethyl)phosphinate is stirred at room temperature for 4 hours. NMR measurements confirm the completeness of the reaction. The residue is dried for 30 minutes at 90° C. under a vacuum of 13.3 Pa, giving 1.67 g of 1-butylpyridinium bis(pentafluoroethyl)phosphinate as a liquid. The yield is virtually quantitative.

¹H NMR (reference: TMS; CD₃CN), ppm: 0.95 t (CH₃); 1.37 m (CH₂); 1.95 m (CH₂); 4.56 t (CH₂); 8.03 m (2CH); 8.52 t,t (CH); 8.83 d (2CH); ³J_{H,H}=7.3 Hz; ³J_{H,H}=7.5 Hz; ³J_{H,H}=7.8 Hz; ³J_{H,H}=5.7 Hz; ⁴J_{H,H}=1.2 Hz.

¹⁹F NMR (reference: CCl₃F —internal; CD₃CN), ppm: −80.2 s (2CF₃); −124.9 d (2CF₂); ²J_{P,F}=66 Hz.

³¹p NMR (reference: 85% H₃PO₄—external; CD₃CN), ppm: −2.5 quin.; ²J_{P,F}=66 Hz.

1. Process for the preparation of onium salts with dialkylphosphate, dialkylphosphinate or (O-alkyl)alkyl- or alkylphosphonate anions by reaction of an onium halide with a trialkyl phosphate, alkyl dialkylphosphinate, dialkyl alkylphosphonate or trialkylsilyl ester or mixed alkyl trialkylsilyl ester of phosphoric, dialkylphosphinic or alkylphosphonic acid.

2. Process according to claim 1, characterised in that, for the synthesis of dialkylphosphate salts, an onium halide is reacted with a trialkyl phosphate or trialkylsilyl ester or mixed alkyl trialkylsilyl ester of phosphoric acid.

3. Process according to claim 1, characterised in that, for the synthesis of dialkylphosphinate salts, an onium halide is reacted with an alkyl dialkylphosphinate or trialkylsilyl ester or mixed alkyl trialkylsilyl ester of dialkylphosphinic acid.

4. Process according to claim 1, characterised in that, for the synthesis of (O-alkyl)alkyl- or alkylphosphonate salts, an onium halide is reacted with a dialkyl alkylphosphonate or trialkylsilyl ester or mixed alkyl trialkylsilyl ester of alkylphosphonic acid.

5. Process according to claim 1, characterised in that the halide is a phosphonium halide, thiouronium halide, guanidinium halide or halide with a heterocyclic cation.

6. Process according to claim 1, characterised in that the halide conforms to the formula (1)
[PR₄]⁺Hal⁻ (1),
where
Hal denotes Cl, Br or I and
R in each case, independently of one another, denotes
H, where all substituents R cannot simultaneously be H,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more R may be partially or fully substituted by —F, but where all four or three R must not be fully substituted by F,
and where, in the R, one or two non-adjacent carbon atoms which are not in the α- or ω-position may be replaced by atoms and/or atom groups selected from the group —O—, —S—, —S(O)— or —SO₂—.

7. Process according to claim 1, characterised in that the halide conforms to the formula (2)
[(R¹R²N)—C(═SR⁷)(NR³R⁴)]⁺Hal⁻ (2),
where
Hal denotes Cl, Br or I and
R¹ to R⁷ each, independently of one another, denote hydrogen or CN, where hydrogen is excluded for R⁷,
straight-chain or branched alkyl having 1 to 20 C atoms,
straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more of the substituents R¹ to R⁷ may be partially or fully substituted by —F, but where all substituents on an N atom must not be fully substituted by F,
where the substituents R¹ to R⁷ may be bonded to one another in pairs by a single or double bond
and where, in the substituents R¹ to R⁷, one or two non-adjacent carbon atoms which are not bonded directly to the heteroatom and are not in the ω-position may be replaced by atoms and/or atom groups selected from the group —O—, —S—, —S(O)— or —SO₂—.

8. Process according to claim 1, characterised in that the halide conforms to the formula (3)
[C(NR¹R²)(NR³R⁴)(NR⁵R⁶)]⁺Hal⁻ (3),
where
Hal denotes Cl, Br or I and
R¹ to R⁶ each, independently of one another, denote hydrogen or CN,
straight-chain or branched alkyl having 1 to 20 C atoms, straight-chain or branched alkenyl having 2-20 C atoms and one or more double bonds,
straight-chain or branched alkynyl having 2-20 C atoms and one or more triple bonds,
saturated, partially or fully unsaturated cycloalkyl having 3-7 C atoms, which may be substituted by alkyl groups having 1-6 C atoms,
where one or more of the substituents R¹ to R⁶ may be partially or fully substituted by —F, but where all substituents on an N atom must not be fully substituted by F,
where the substituents R¹ to R⁶ may be bonded to one another in pairs by a single or double bond
and where, in the substituents R¹ to R⁶, one or two non-adjacent carbon atoms which are not bonded directly to the heteroatom and are not in the ω-position may be replaced by atoms and/or atom groups selected from the group —O—, —S—, —S(O)— or —SO₂—.

9. Process according to claim 1, characterised in that the halide conforms to the formula (4)
[HetN]⁺Hal⁻ (4)
where
Hal denotes Cl, Br or I and
HetN⁺ denotes a heterocyclic cation selected from the group 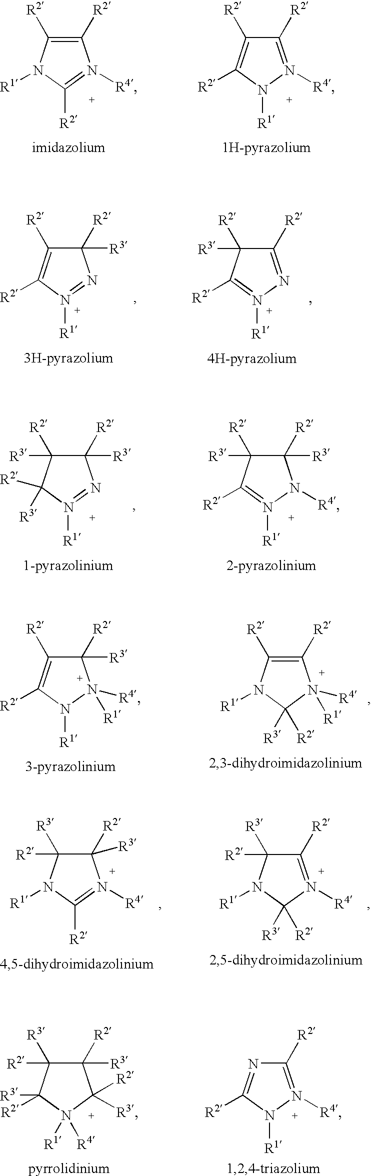 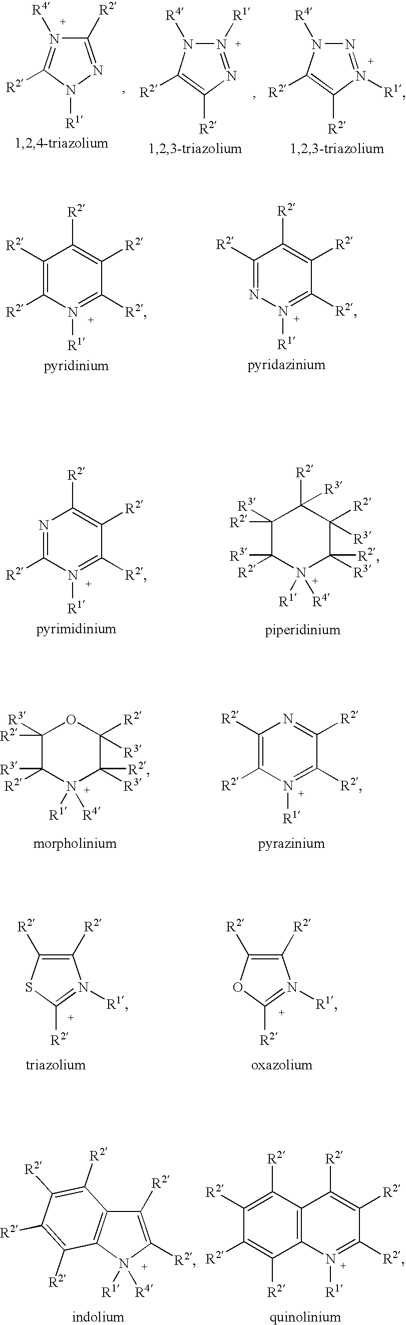 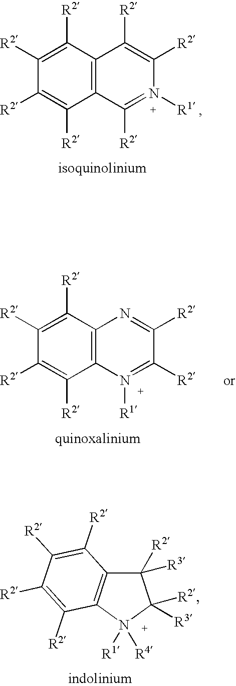
where the substituents
R^{1′} to R^{4′} each, independently of one another, denote hydrogen or CN,
straight-chain or branched alkyl having 1-20 C atoms,
straight-chain or branched alkenyl having 2-20 C atoms and one or more double bonds,
straight-chain or branched alkynyl having 2-20 C atoms and one or more triple bonds,
dialkylamino having alkyl groups having 1-4 C atoms, but which is not bonded to the heteroatom of the heterocycle,
saturated, partially or fully unsaturated cycloalkyl having 3-7 C atoms, which may be substituted by alkyl groups having 1-6 C atoms, or aryl-C₁-C₆-alkyl,
where the substituents R^{1′} and R^{4′} may be partially or fully substituted by F, but where R^{1′} and R^{4′} cannot simultaneously be CN or fully substituted by F, where the substituents R^{2′} and R^{3′} may be partially or fully substituted by halogens or partially substituted by NO₂ or CN
and where, in the substituents R^{1′} to R^{4′}, one or two non-adjacent carbon atoms which are not bonded directly to the heteroatom and are not in the ω-position may be replaced by atoms and/or atom groups selected from the group —O—, —S—, —S(O)— or —SO₂—.

10. Process according to claim 1, characterised in that the reaction of the alkyl esters of phosphoric, dialkylphosphinic or alkylphosphonic acid is carried out at temperatures of 20° C. to 100° C.

11. Process according to claim 1, characterised in that the reaction of the trialkylsilyl esters of phosphoric, dialkylphosphinic or alkylphosphonic acid is carried out at temperatures of 0° C. to 30° C.

12. Process according to claim 1, characterised in that the reaction is carried out without a solvent.

13. Use of the process according to claim 1 for the purification of ionic liquids with dialkylphosphate, dialkylphosphinate, (O-alkyl)alkylphosphonate or alkylphosphonate anions which are contaminated by onium halides.

14. Trialkylsilyl esters of the formula (C₂F₅)₂P(O)OSi(alkyl)₃, (C₃F₇)₂P(O)OSi(alkyl)₃ or (C₄F₉)₂P(O)OSi(alkyl)₃, where the alkyl groups of the trialkylsilyl group can have 1 to 4 C atoms.
